(19) United States
(12) Patent Application Publication
(10) Pub. No.: US 2002/0014023 A1
(43) Pub. Date: Feb. 7, 2002

(54) Adjustable support and methods for shoes

(76) Inventors: Hindy Kellerman Tolwin, Beverly Hills, CA (US); David Kellerman, Santa Barbara, CA (US)

Correspondence Address: Lewis B. Sternfels, Law Offices of Clark D. Gross, Suite 300, 12304 Santa Monica Boulevard, Los Angeles, CA, 90025-2593, US

(21) Appl. No.: 09/917,495

(22) Filed: Jul. 28, 2001

Related U.S. Application Data
(60) Provisional application No. 60/221,321, filed on Jul. 28, 2000.
(60) Provisional application No. 60/253,979, filed on Nov. 29, 2000.

(51) Int. Cl. A61F 5/14

(52) U.S. Cl. 36/155; 36/160

(57) ABSTRACT

An insole (20, 90, 110) provides for adjustable pediatric support characteristics for a user's foot. One or more pairs of stacked rotor and stator resilient elements (34, 36) are supported by the insole. Each pair of resilient elements is characterized by having a greater resiliency when the rotor is oriented along or at 90° with respect to the stator. The rotor is relatively movable with respect to the stator to effect different orientations of the paired rotor and stator and enables the blending of the respective resiliences of the pair and, thereby, for providing the adjustable pediatric support characteristics. A plurality of rotor and stator pairs enables adjustments for different parts of the foot and for differences between an individual's feet.

### CROSS-REFERENCE TO RELATED APPLICATION

This application claims the benefit of U.S. Provisional applications Ser. Nos. 60/221,321 filed Jul. 28, 2000 and No. 60/253,979 filed Nov. 29, 2000.

### BACKGROUND OF THE INVENTION

1. Field of the Invention

The present invention relates to insoles for footwear and, more particularly, to means and methods for adjusting and varying the degree and resiliency of support for one or both feet.

2. Description of Related Art and Other Considerations

The narrow part of the sole of a shoe under the instep, called the shank piece, ideally should flex to a certain degree as one walks or runs, to avoid restraining the natural functioning of the foot. Because no one foot is exactly like another, when an adjustment is required, the adjustment must be tailored to the specific foot under consideration. Such tailoring may be obtained by an existing commercially available insole or by a specially constructed insole, such as prescribed or formulated by a podiatrist or other professional. The latter construction may be expensive and, therefore, not a viable option to many. Independent adjustment for differently formed feet or different foot problems between the feet is not easily and possibly inexpensively obtainable. Regardless, any adjustment of the bending movement in one or more zones within a shoe is not easily obtainable.

### SUMMARY OF THE INVENTION

These and other problems and considerations are successfully addressed and overcome by the present invention. An insole comprises one or more internally supported pairs of stacked resilient elements. Each supporting element has at least two axes intersecting one another and is characterized by having a greater resiliency along a first of the axes than along a second of the axes. The elements are relatively movable with respect to one another to enable relative movement of their respective axes to vary the combined resiliency between the elements and, thereby to provide adjustable podiatric support characteristics in the insole.

Preferably, each element is configured as a spring-like disc, with one being stationary with respect to the remainder of the insole, to thereby act as a stator, and the other being rotatable with the stator. Also preferably, the latter disc, or a rotor, is circular, so that it may be retained within circular confines in the insole and be turned, such as by a screwdriver type tool which may be inserted through an appropriate opening in the bottom of the insole so as to engage a slot formed in the rotor. The use of slots in the rotor forms a visible method of visibly determining the orientation of the rotor with respect to the stator.

The spring-like disc may take such configurations as a thin spring flat leaf or a spider, and the components of the disc may also be differently formed so as to provide a uniformly or differently programmed resiliency. For example, a circular disc containing parallel strips secured within a peripheral supporting ring, provides different resiliency among the several shorter and longer strips, if the strips have the same widths; however, by tailoring the widths of the several individual strips, their resilient characteristics may be made uniform, or otherwise programmed, as desired.

Several advantages are afforded by the present invention. The resilient characteristics of an insole are easily and relatively inexpensively obtained. The insole as a whole or in part may be quickly and simply tuned or tailored to the individual foot by the professional or by the individual. Tuning may be effected independently for the two feet, and in as simple a manner as by a screwdriver or like tool. Orientation of the resiliency/stiffness characteristics are made visible. The size of the adjustable elements permits use of the present invention in a wide variety of shoes, whether of a fashionable or work version, or a low or high-heel type.

Reduction to some extent is possible of tendinitis or related problems due to wearing higher heel shoes by using the invention with such shoes and setting the adjustments on the very low side, as a conceivable method to allow the shanks of the legs to bend more easily, thus resulting in exercising more and reducing the tendon brittleness increase with time in wearing high heels. Due to anatomical differences of feet or legs for many people, individual right and left side shank-piece adjustment will be beneficial.

A low cost, reliable and functional method is provided for a fit in a thin insole area, where no visibility for styling considerations is important.

Other aims and advantages, as well as a more complete understanding of the present invention, will appear from the following explanation of exemplary embodiments and the accompanying drawings thereof.

### BRIEF DESCRIPTION OF THE DRAWINGS

FIG. 1 is a top plan view of a first embodiment of an insole embraced by the present invention, showing interior elements thereof in phantom;

FIG. 2 is a bottom plan view of the FIG. 1 embodiment, portraying an entry way to interior elements, which are shown in phantom;

FIG. 3 is an enlarged view of a portion of FIG. 2;

FIG. 4 is a view, in cross-section, of the first embodiment taken along line 4-4 of FIG. 3;

FIG. 5 is a view of an interior portion of the first embodiment taken at a first level or layer to depict a stationary resilient member or stator;

FIG. 6 is a view of an interior portion of the first embodiment taken at a second level or layer to depict a stationary resilient member or rotor;

FIG. 7 is an enlarged view of the central section illustrated in FIG. 6 and showing a first orientation between the rotor and the stator to produce one of the many adjustably resilient stator-to-rotor combinations provided by the present invention;

FIG. 8 is a view similar to that depicted in FIG. 7, but showing a second orientation between the rotor and the stator, in which the rotor is turned 90° with respect to that of the stator, to produce another adjustably resilient combination;

FIGS. 9-13 illustrate, in plan and cross-sectional views, different spring configurations of the stator and/or rotor useful in carrying out the concepts of the present invention;

FIG. 14 is a second embodiment of the present invention depicting different positionings of three stator-to-rotor combinations for enabling specific adjustably resilient combination to different parts of the foot;

FIG. 15 is a view of an assembly of four spring elements oriented along parallel axes to provide a maximum stiffness or resilient characteristic to the insole, as being dependant upon whether all their parallel axes are parallelly or orthogonally disposed with respect to the underlying spring element; and

FIG. 16 is a view of the four FIG. 15 spring elements differently rotated to dispose their respective axes in a differently angled orientation thereamongst, so as to illustrate varying degrees of stiffness and resiliency for application to different parts of the foot.

### DETAILED DESCRIPTION OF THE PREFERRED EMBODIMENTS

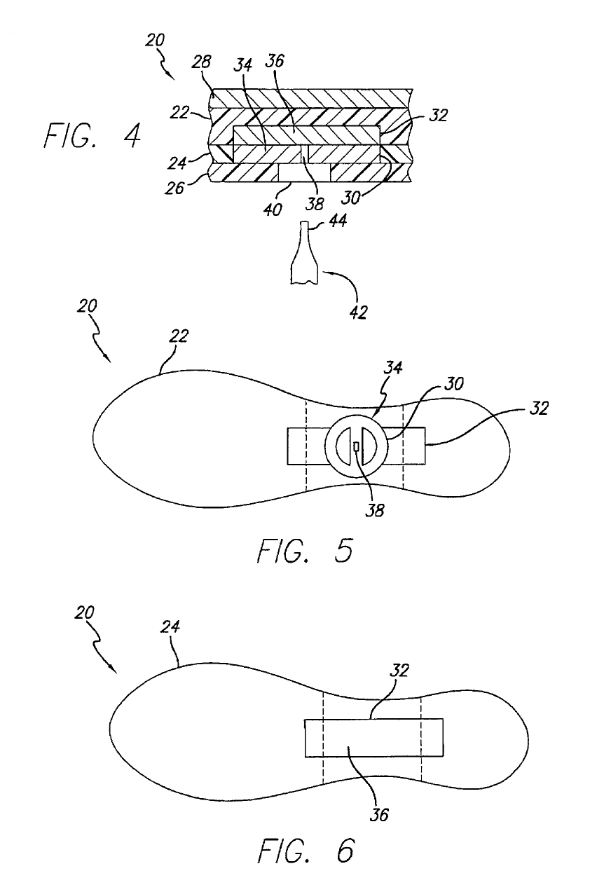

As illustrated in FIGS. 1-8, which related to a first embodiment of the present invention, an insole 20 comprises a plurality of layers, as best shown in FIG. 4, such as layers 22, 24, 26 and 28, which are secured together to provide a complete unit for insertion into the shoe of and for support of an individual. Layer 26, as the bottom layer, may comprise a soft cushion material. Layer 28, as the top layer, may comprise a finishing layer. Layers 26 and 28 therefore form supporting outer layers for intermediate layers 22 and 24.

Intermediate layers 22 and 24 include respective openings 30 and 32 for receipt of a first and second resilient elements 34 and 36. Together, first and second elements form a pair of stacked first and second resilient elements which are supported by a supporting medium comprising at least layers 22 and 24. For the embodiment illustrated in FIGS. 1-8, resilient element 36 is fixed with respect to the supporting medium of layers 22 and 24, and may be referred to as a stator. Stator element 36 is configured to have axes 36x and 36y disposed normally with respect to one another. Resilient element 34, however, is disposed to be moveable, in particular rotatable, within opening 30 and, therefore, with respect to the supporting medium. Accordingly, resilient element 34 may be referred to as a rotor. Rotor element 34 is configured to have axes 34x and 34y disposed normally to one another.

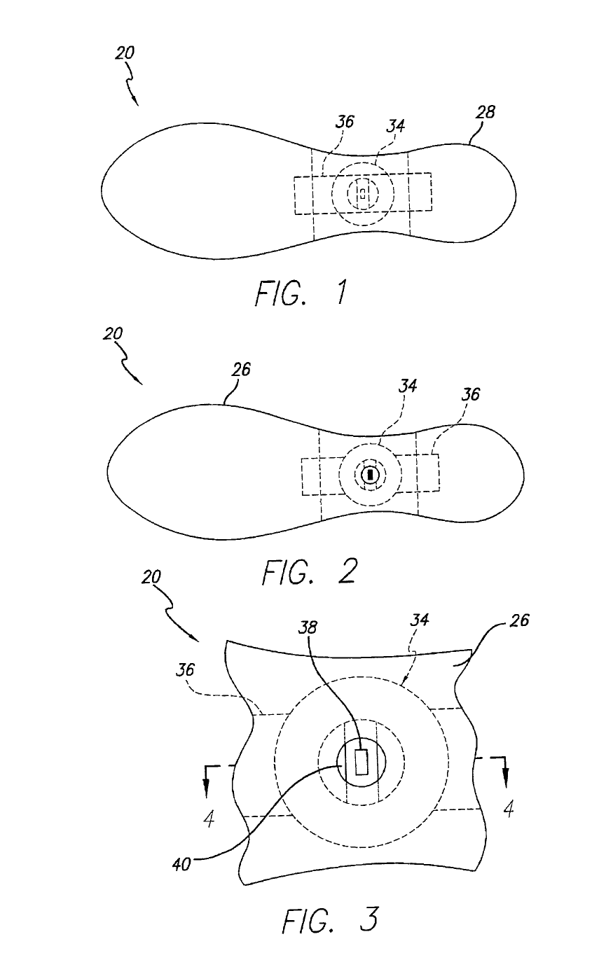

To enable turning of rotor 34, a slot 38 (see FIGS. 2-4 and 6-8) is formed at its center, and an opening 40 is provided in bottom layer 26 to afford access to slot 38. As illustrated in FIG. 4, a screwdriver 42 or like tool, having a tip 44 which is shaped similarly to that of the slot, is insertable through opening 40 and, therefore, can engage the slot and turn resilient rotor element 34. Slot 38 also provides a visible indicator as to the orientation of rotor element 34 with respect to fixed stator 36. The assembly is so enclosed that it is made water tight, with the only exposed surfaces being the slot and immediately adjacent rotor area; preferably, a rubber washer and/or other protection is placed over and/or around the slot and its area during assembly of the insole. The assembly may also contain suitable thin gasket or washer layers which can flex while providing an effective seal against entry of moisture or other liquid.

Should it be found that any movement between the elements tend to be too tight, a Teflon, silicone, or similar coating may be inserted or otherwise employed. If. However, there is too mush slippage, a thin rubber washer or suitable material can be used.

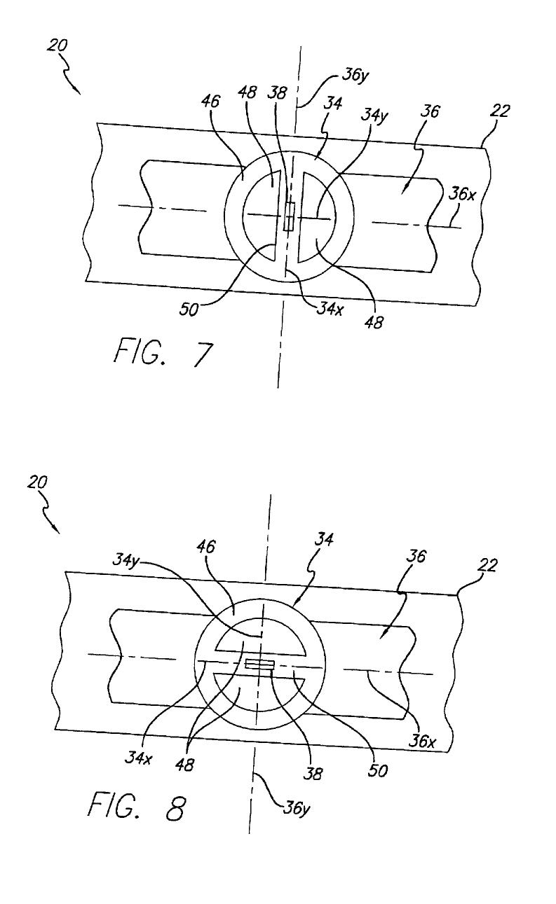

As best depicted in FIGS. 7 and 8, resilient rotor element 34 is shown as comprising a circular thin disc formed, for example, of spring steel, stainless, or any other suitable material. Stator element 36 may take any configuration, and is depicted as a rectangular thin sheet, also formed, for example, of spring steel, stainless, or any other suitable material. The combined rotor and stator elements may have a total thickness of approximately 0.06″ to 0.1″ which will fill within the thickness range of typical suitable shanks of insole 20 of approximately 0.15″ to 0.3″ thick.

The rotor element includes a closed periphery 46 having open spaces 48 therein which form a link 50 connecting opposed sides of the periphery. As depicted, link 50 lies on axis 34x and, because of the existence of the link and the absence of any connection dissected by axis 34y, rotor element 34 is more resilient about axis 34y than about axis 34x.

Stator element 36 comprises a single thin spring flat leaf which cannot move but can spring to some degree, up and down, as one walks or runs due to the change in alternately placing all the body weight on the heel and toes of the foot as they cause the shoe to meet the ground.

When the rotor and stator elements are aligned so that their respective axes 34y and 36x are aligned, as shown in FIG. 7, the combined resiliency due to this orientation, is the greatest. When the rotor and stator elements are aligned so that their respective axes 34x and 36x are aligned, as shown in FIG. 8, the combined resiliency due to this orientation, is the least.

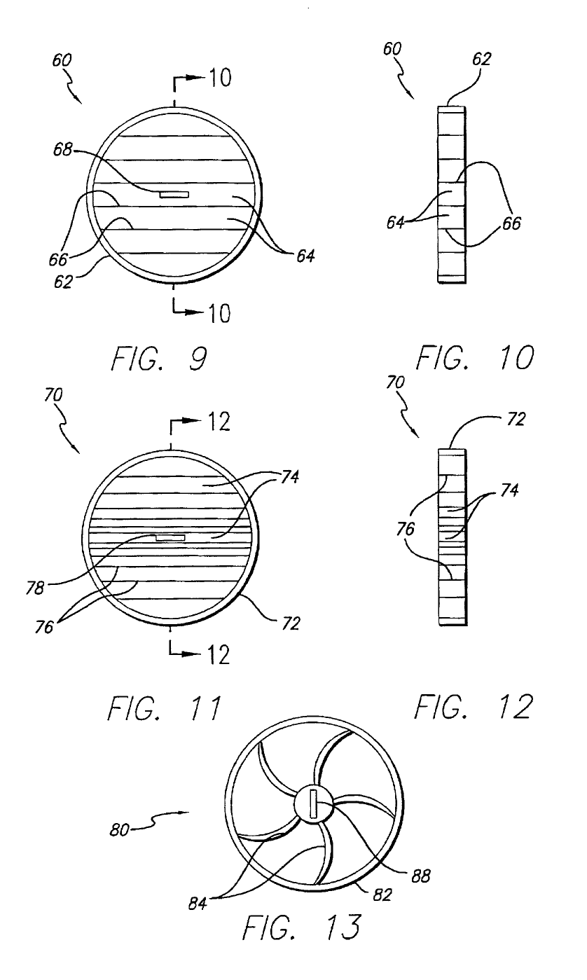

Both the stator and the rotor, in particular the rotor, may be configured as depicted in FIGS. 9-13. Rotor element 60 of FIGS. 9 and 10 is configured as a disc and comprises an annular periphery 62 and a plurality of leaves 64 of uniform widths, formed by slitting the material from which the rotor disc is fabricated. A slot 68 is formed in the center leaf to enable turning of the rotor with respect to the stator. Because the lengths of the leaves are not equal, the resiliency characteristics of this rotor disc vary across its diameter. Should such resiliency characteristics be desired to be uniform or otherwise programmed, a rotor disc 70, as illustrated in FIGS. 11 and 12 may be employed, having a periphery 72 supporting a plurality of leaves 74 whose widths vary according to the program. Leaves 74 are formed by slits 76. As in the case of the prior rotor, a slot 78 is placed in the centrally located leaf. Other rotor element configurations may be employed, as desired. An example thereof is depicted in FIG. 13, in which a rotor 80 comprises a periphery 82 and a spider-like plurality of leaves 84 supported on a central hub 86, in which a turn-effecting slot 88 is located.

Reference is now made to FIG. 14 which depicts an insole 90 housing three spring elements 92, 94 and 96 in its insole shank 98, in which each in combination with its mating portion of the stator, or three individual stators if desired, to provide three pairs of stacked first and second resilient elements supported by insole 90 as the supporting medium. The stacked pairs including spring elements 92, 94 and 96 are respectively positioned to form adjustments for the respective medial side, center and outside of the shank. Each spring element is provided with its slot, generally identified by indicium 100, for individual adjustment of the respective paired rotor-stator elements by proper individual orientation thereof. This embodiment is useful, for example, to advantage for a two or three adjustment of the shank is for toeing the shoes in or out and, therefore, it will be possible to adjust and correct for related abnormal foot conditions.

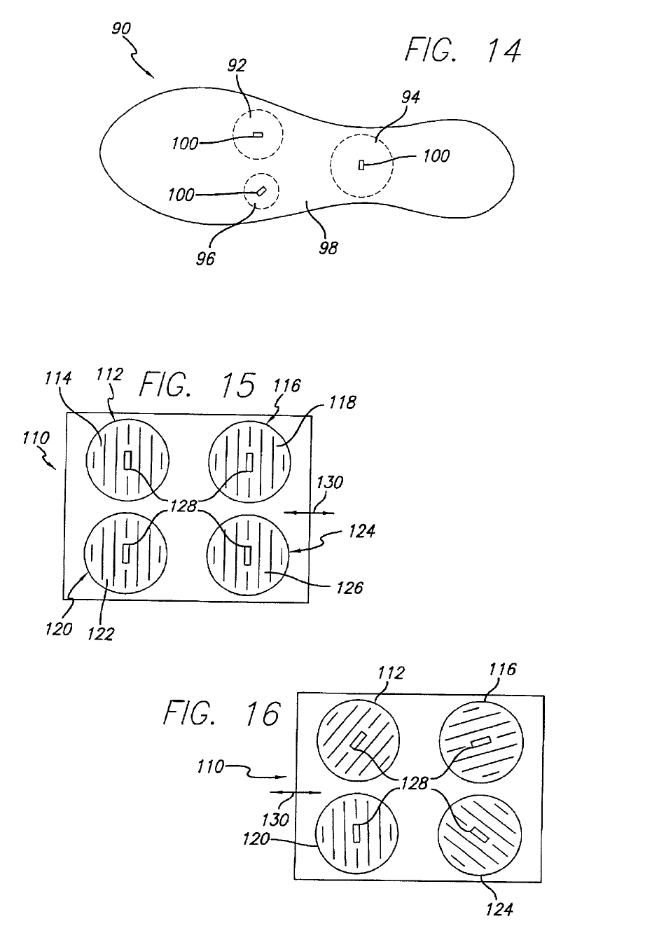

FIGS. 15 and 16 illustrate a further embodiment of an insole 110 depicting an assembly of four spring element pairs in which the rotor resilient spring elements of each are shown, comprising an element 112 and its parallelly disposed leaves 114, an element 116 and its parallelly disposed leaves 118, an element 120 and its parallelly disposed leaves 122 and an element 124 and its parallelly disposed leaves 126. Each rotor spring element is paired with its stator element which may be a portion of a leaf spring, such as leaf spring 36 of FIGS. 1-8, or an individual piece. Slots, as generally identified by indicium 128, are engageable with a tool, such as the tip of a screwdriver, for turning the individual rotor elements into an orientation to provide a desired degree of resiliency or stiffness.

The orientations of the four elements 112, 116, 120 and 124 along their parallel axes provide maximum stiffness or resilient characteristics to the insole, as being dependant upon whether all their parallel axes are parallelly or orthogonally disposed with respect to the underlying spring element. For the toe and heel portions of insole 110 extending in the direction of double-headed arrow line 130, the orientations of these four elements produce a maximum stiffness in the insole. If they were rotated 90° with respect to that shown in FIG. 15, these orientations would produce maximum resiliency in the insole.

When the four rotor elements of FIG. 15 are turned to those depicted in FIG. 16, the four spring elements provide different spring characteristics. Specifically, element 112 provides about a 70% stiffness direction to the left, element 116 provides a minimum stiff direction, element 120 provides a maximum stiffness direction, and element 124 provides about a 70% stiffness direction toward the right.

The embodiment illustrated in FIGS. 15 and 16 can be used to provide the benefits of protection from excessive shock to the heel and forefoot sections of the feet for walking, running and engagement in active sports.

The quadrature preferred arrangement can be made to be about a 1½″ to 3″ square assembly with a total thickness of approximately 0.063″ to 0.2″ for typical shank embedment. The four adjustment slots can be located on the bottom of the shank or on the bottom inside of the shoe or other footwear. It is evident that the four zones will be able to achieve variations in flexibility. Discrete effects on supination, pronation and other variations in support of arches, and other areas of the feet is made possible with the present invention. By varying the spring temper and material used for the spring plates, additional choices of lighter to heavier duty models can be made.

The present invention may use a shorter or a full length metal insole, which is shaped flat, thin, or have a spring-back. The insole may rest on the upper inside of the shoe, and be removable and re-insertable for adjustable angle positioning. There can be several areas of location such as one or more discs in the medial arch area and in the metatarsal areas. Various pads, attachments and the like can be placed over this spring-back insole to provide soft adjustable cushioning as well as a flat spring-back, and with adjustable intensity and variable direction of lateral forces. Attachment of pads may be effected by hook and loop attachment systems, and connected to provide lateral stability. The metal flat plates can be made of thicker or thinner sheet materials to provide heavier or lighter zones of plantar support and spring-back action. The areas can have plates of stiffer materials or more dead soft temper materials. The materials can be spring steel, beryllium copper or a host of other materials, such as fiberglass or carbon fibre. The flat discs can be smaller or larger in diameter to suit specific plantar aspect areas. Thus, the present inventive enhanced design affords a totally adjustable spring-back and shock absorber insole which can be used for virtually any type of footwear.

The spring-back section can be made relatively thin and, together with the cushioning section, can total from 0.1″ for standard use to about 0.25″ for heavy duty work or athletic use. For improved visibility of the round disc with serrated variable and directional areas, the spring-back assembly can have a bottom layer about 0.004″ thick. The discs are about 0.01″ thick and the top layer is a clear sheet of approximately 0.006″ polypropylene or other suitable material, with the bottom layer laminated with about a 0.0005″ layer of polypropylene or other suitable material. The holes in the center sheet can be loaded with the discs and the upper clear plastic sheet can be permanently bonded to the thin 0.0005″ plastic layer on the lower sheet. The result can be a thin, approximately 0.0006″+0.0005″+0.004″ or 0.015″ typical thickness. These dimensions can be reduced to total 0.01″ and provide a thin, rugged, flat, flexible, and adjustable, unique plantar aspect, protective foot exerciser and comfort device.

The metal plates can provide a cooling effect as they can conduct heat away from the warm heat generating areas.

Although the invention has been described with respect to particular embodiments thereof, it should be realized that various changes and modifications may be made therein without departing from the spirit and scope of the invention.

What is claimed is:

1. An insole having adjustable podiatric support characteristics for a user's foot, comprising:
a supporting medium;
at least one pair of stacked first and second resilient elements supported by said supporting medium,
each said resilient element having at least two axes intersecting one another and being characterized by having a greater resiliency along a first of the axes with respect to that along a second of the axes,
said elements being relatively movable with respect to one another to effect different orientations of and between their respective axes for blending the resiliencies of said elements and, thereby, for providing the adjustable podiatric support characteristics.

2. An insole according to claim 1 in which at least said first of said resilient elements comprises a disc having a closed periphery and spring-like components held by and contained within said periphery.

3. An insole according to claim 2 in which said components have a spider-like configuration.

4. An insole according to claim 2 in which said components have a flat leaf-like configuration.

5. An insole according to claim 2 in which said flat leaf-like configured components comprise parallel strips lying parallel to one of the axes and having given lengths and widths, and in which the lengths and widths are so interrelated as to provide a programmed resiliency characteristics to said disc.

6. An insole according to claim 5 in which said closed periphery is circular to impart said parallel strips with different lengths, and in which the widths are equal so as to provide a varying resiliency along one of the axes of said disc.

7. An insole according to claim 5 in which said closed periphery is circular to impart said parallel strips with different lengths, and in which the widths are varied so as to provide a varying resiliency along one of the axes of said disc.

8. An insole according to claim 7
in which said disc is provided with a slot of irregular shape, said medium includes an enclosure about said elements, said enclosure includes an opening providing access to said slot, and said second of said elements is stationary with respect to the remainder of the insole, and
further including a tool having an end shaped corresponding to that of the slot for insertion through the enclosure opening and into engagement with the slot for rotation of said first element disc with respect to said second element.

9. An insole according to claim 8 in which said second element is shaped as a disc, similarly to said first element disc.

10. An insole according to claim 9 further including at least one additional pair of stacked first and second resilient elements which are supported by said supporting medium and which are similarly configured as said first-mentioned pair of stacked resilient elements, wherein said pairs of stacked resilient elements are positioned in different locations in said medium for providing the adjustable podiatric support characteristics to different parts of the user's foot.

11. A method for adjusting the podiatric support characteristics for a user's foot, comprising the steps of:
stacking, in a supporting medium, at least one pair of first and second resilient elements, each having at least two axes intersecting one another, and being characterized by having a greater resiliency along a first of the axes with respect to that along a second of the axes,
moving the elements relatively with respect to one another to effect different orientations of and between their respective axes for blending their resiliencies and, thereby, for providing the adjustable podiatric support characteristics.

12. A method according to claim 11 further comprising the steps of utilizing peripherally supported spring-like components to provide the resiliencies, and varying the dimensions of the spring-like components in at least a first of the elements to provide programmed resiliency characteristics thereto.

13. A method according to claim 12 in which the spring-like components have a flat leaf-like configuration of parallel strips lying parallel to one of the axes and having given lengths and widths, further comprising the step of interrelating the lengths and widths of the spring-like components as to provide programmed resiliency characteristics to the first element.

14. A method according to claim 13 in which the closed periphery is circular to impart the parallel strips with different lengths, wherein said interrelating step comprises the step of making the widths equal so as to provide a varying resiliency along one of the axes of the first element.

15. A method according to claim 13 in which the closed periphery is circular to impart the parallel strips with different lengths, wherein said interrelating step comprises the step of making widths varied so as to provide a varying resiliency along one of the axes of the first element.

16. A method according to claim 13 in which the closed periphery is circular and the second resilient element is stationary with respect to the supporting medium, further comprising the steps of:
providing the first element with an indentation, and the supporting medium with an opening providing access to the indentation; and
utilizing a tool having an end shaped corresponding to that of the indentation for insertion through the opening and into engagement with the indentation for rotation of the first element with respect to the second element.

17. A method according to claim 9 further including the steps of providing at least one additional pair of stacked first and second resilient elements which are supported by the supporting medium and which are similarly configured as the first-mentioned pair of stacked resilient elements, and positioning all the pairs of stacked resilient elements in different locations in the medium for providing the adjustable podiatric support characteristics to different parts of the user's foot.
